(19) United States
(12) Patent Application Publication
(10) Pub. No.: US 2010/0233145 A1
(43) Pub. Date: Sep. 16, 2010

(54) COMPOSITION USED AS A NUTRITIONAL SUPPLEMENT AND MEDICATION FOR THE TREATMENT OF DEGENERATIVE VISUAL DISORDERS AND INFLAMMATION

(75) Inventor: Theodorus van Veen, Lund (SE)

Correspondence Address: BARNES & THORNBURG LLP, Suite 1150, 3343 Peachtree Road, N.E., Atlanta, GA, 30326-1428, US

(73) Assignee: APO PHARMACEUTICALS INTERNATIONAL B.V., Volendam (NL)

(21) Appl. No.: 12/279,743

(22) PCT Filed: Feb. 16, 2007
(86) PCT No.: PCT/NL2007/050064

§ 371 Date: Jun. 2, 2010

(30) Foreign Application Priority Data
Feb. 16, 2006 (NL) 2000008

(51) Int. Cl. A61K 31/385; A61P 29/00; A61P 27/02

(52) U.S. Cl. 424/94.1; 514/440

(57) ABSTRACT

A composition and a dosing unit which can be used as nutritional supplement or as medication for the treatment of retinitis pigmentosa (RP). The composition comprises a combination of lutein, zeaxanthin, glutathione and alpha lipoic acid, optionally supplemented with an extract of Lucium Barbarum Lynn. This composition inhibits the death of photoreceptors as a consequence of RP, whereby the visual faculties of the patient are retained for a longer time. The composition is also useful as an anti-inflammatory drug.

The invention relates to a composition. The invention also relates to a package comprising the composition. The invention also relates to the composition as nutritional supplement, as medication for treating degenerative visual disorders, and as an anti-inflammatory medicament.

Retinitis Pigmentosa (RP) is a heterogeneous group of hereditary degenerative retinal disorders ultimately resulting in blindness. Examples of disorders falling within the RP group are autosomal dominant retinitis pigmentosa, autosomal recessive retinitis pigmentosa and X-related retinitis pigmentosa (XLRP). An estimated 1 in 3000 people in the industrialized world suffer from a form of RP (Kalloniatis M, Fletcher E L (2004) Retinitis pigmentosa: understanding the clinical presentation, mechanisms and treatment options. Clin. Exp. Optom. 87: 65-80). This group of disorders has the common feature of a progressive loss of rod photoreceptors in the eye, which results in a poorer adaptability to light intensity, night blindness and the development of tunnel vision. Degeneration of the cone photoreceptors also becomes noticeable in the longer term, this resulting in a reduced contrast and colour perception. The increasing degeneration of the photoreceptors can ultimately lead to blindness. The death of the photoreceptors cells normally takes place in RP via apoptosis. At this moment no effective treatments for RP are known.

The invention has for its object to provide a means for the treatment of Retinitis Pigmentosa.

The invention provides for this purpose a composition comprising pharmaceutically active quantities of lutein, zeaxanthin, glutathione and alpha lipoic acid. The combination of these active components inhibits the degeneration of the photoreceptors of RP-patients. To this end these substances must be administered daily in active quantities, for instance as nutritional supplement. The positive effect of the carotenoids lutein and zeaxanthin on some eye disorders is per se known. The combination of these two substances together with glutathione and alpha lipoic acid is found to have a synergistic effect. Glutathione is the usual name for L-glutathione, abbreviated to GSH.

Both the oxidized and reduced form of glutathione can be used. Alpha lipoic acid is also known as thioctic acid. The systematic name of zeaxanthin is (3R, 3′R)-β,β-carotene-3,3′-diol, that of lutein is.: (3R, 3′R, 6′R)-(3,c-carotene-3,3′-diol. In addition to the active ingredients, the composition can also comprise an appropriate pharmaceutically acceptable excipient. It is possible here to envisage solid excipients such as magnesium stearate, talc or liquid diluents such as water and/or alcohol. The composition can also comprise pharmaceutically acceptable additives such as emulsifiers, surfactants and/or conserving means.

It is advantageous if the composition comprises at least 1 mg lutein, at least 1 mg zeaxanthin, at least 20 mg glutathione, and at least 5 mg alpha lipoic acid. Such quantities are active, although it is often necessary to use a multiple of these quantities in order to achieve the desired effect. The daily dosage required can be split up into multiple dosing units which can be ingested at different times during the day.

In a preferred embodiment the composition comprises from 1 to 40 mg lutein, from 1 to 40 mg zeaxanthin, from 20 to 800 mg glutathione, and from 5 to 300 mg alpha lipoic acid. Compositions within these weight ratios of the ingredients have a particularly good effect. The upper values correspond with the recommended daily dosage for an average person. The daily dosage can be divided up into multiple units, such as pills or capsules.

It is recommended that the composition comprises from 10 to 40 mg lutein, from 10 to 40 mg zeaxanthin, from 200 to 800 mg glutathione, and from 50 to 300 mg alpha lipoic acid. Use of these relatively large quantities is found to give a particularly good effect. Quantities larger than the indicated upper limits can in principle provide an even better effect, but in most cases this improvement is no longer significant. Between 400 and 800 mg glutathione is preferably present: this is found to provide a particularly good effect.

The composition more preferably comprises from 15 to 25 mg lutein, from 15 to 25 mg zeaxanthin, from 180 to 220 mg glutathione, and from 90 to 110 mg alpha lipoic acid. With such a composition a particularly good effect is combined with economic use of the ingredients.

It is advantageous if lutein, zeaxanthin, glutathione and alpha lipoic acid are present in the relative weight ratio of 1 to 4 units of weight of lutein, of 1 to 4 units of weight of zeaxanthin, of 16 to 80 units of weight of glutathione, and of 5 to 30 units of weight of alpha lipoic acid. The optimum synergistic effect of the ingredients is realized at these weight ratios. Lutein and zeaxanthin are preferably present here in a relative weight ratio of between 1:2 and 2:1. It is also advantageous if glutathione is present in a weight ratio of between 40 and 80 units of weight. Such a ratio is found to provide a particularly good effect.

In a preferred embodiment the composition also comprises an active quantity of Lucium Barbarum Lynn extract. The addition of such an extract provides an unexpected improvement in the effect of the combination. Lucium Barbarum Lynn extract (LBL-extract) is an extract from the fruit of Lucium Barbarum Lynn, which is also known under the name ‘Wolfberry’. Such extracts are commercially available. The best effect is obtained if an extract is used which comprises at least 90% by weight of polysaccharides. The polysaccharide content of an LBL-extract can for instance be determined by UV spectrophotometry.

It is advantageous if the composition comprises at least 20 mg Lucium Barbarum Lynn extract. The composition more preferably comprises at least 1 mg lutein, at least 1 mg zeaxanthin, at least 20 mg glutathione, at least 5 mg alpha lipoic acid and at least 20 mg Lucium Barbarum Lynn extract.

The composition preferably comprises from 20 to 1000 mg Lucium Barbarum Lynn extract. The composition more preferably comprises from 10 to 40 mg lutein, from 10 to 40 mg zeaxanthin, from 200 to 800 mg glutathione, from 50 to 300 mg alpha lipoic acid and from 20 to 1000 mg Lucium Barbarum Lynn extract.

It is advantageous if the composition comprises lutein, zeaxanthin, glutathione, alpha lipoic acid and LBL-extract in the following relative weight ratio: from 1 to 4 units of weight lutein, from 1 to 4 units of weight zeaxanthin, from 16 to 80 units of weight glutathione, from 5 to 30 units of weight alpha lipoic acid, and from 20 to 100 units of weight Lucium Barbarum Lynn extract.

The invention also provides a composition according to the invention for use as nutritional supplement. Use of the composition according to the invention can prevent and/or inhibit the degeneration of photoreceptor cells of the eye. The nutritional supplement is preferably ingested daily. Use of such a nutritional supplement is particularly advantageous for people with a genetic tendency toward the development of retinitis pigmentosa. The effect is preventive and prevents or delays degeneration of the eyesight occurring for instance due to age-related macula degenaration.

Apart from the preventive effect, the composition according to the invention is also usefull as a medication for the treatment of degenerative visual disorders, in particular retinitis pigmentosa, diabetic retinopathy, macular degeneration and cataracts. Although these diseases are not cured, the degenerative effect on eyesight that is typical for these diseases can be slowed down or in some cases halted by daily ingestion of an effective amount of the composition according to the invention.

In particular, the composition has been found to be usefull as a medication for the treatment of retinitis pigmentosa.

The invention also provides the use of a composition according to the invention for treating retinitis pigmentosa. The use of the composition according to the invention can prevent and/or inhibit the degeneration of photoreceptor cells of the eye. The composition according to the invention has a favourable effect on the development of the clinical symptoms in different forms falling within the collective name retinitis pigmentosa. These include the genetically classifiable disorders of autosomal dominant retinitis pigmentosa, autosomal recessive retinitis pigmentosa and X-linked retinitis pigmentosa (XLRP), although genetically more complex disorders not falling within these classifications also belong to the group of disorders which can be treated with a composition according to the invention. The composition slows degeneration of the eyesight.

Surprisingly it was found that the composition according to the invention is also useful as an anti-inflammatory medication. Persons treated with the composition according to the invention for treating retinal degeneration noted that as a side-effect, pains and complaints related to inflammation were diminished. This indicates that a composition comprising pharmaceutically active quantities of lutein, zeaxanthin, glutathione and alpha lipoic acid is useful medicament against inflammatory diseases. In particular improvement of conditions related to arthritis were observed in patients.

The invention further provides a package comprising a composition according to the invention. The package may contain multiple dosing units such as pills or capsules. The invention also provides a dosing unit comprising a composition according to any of the foregoing claims. The dosing unit can have any suitable known administering form, wherein oral forms of administration such as pills and capsules can be particularly envisaged.

The invention will now be elucidated on the basis of the following examples.

FIG. 1 shows the development of RP in TUNEL-stained retinas of CH3H wild type and rd/rd mice.

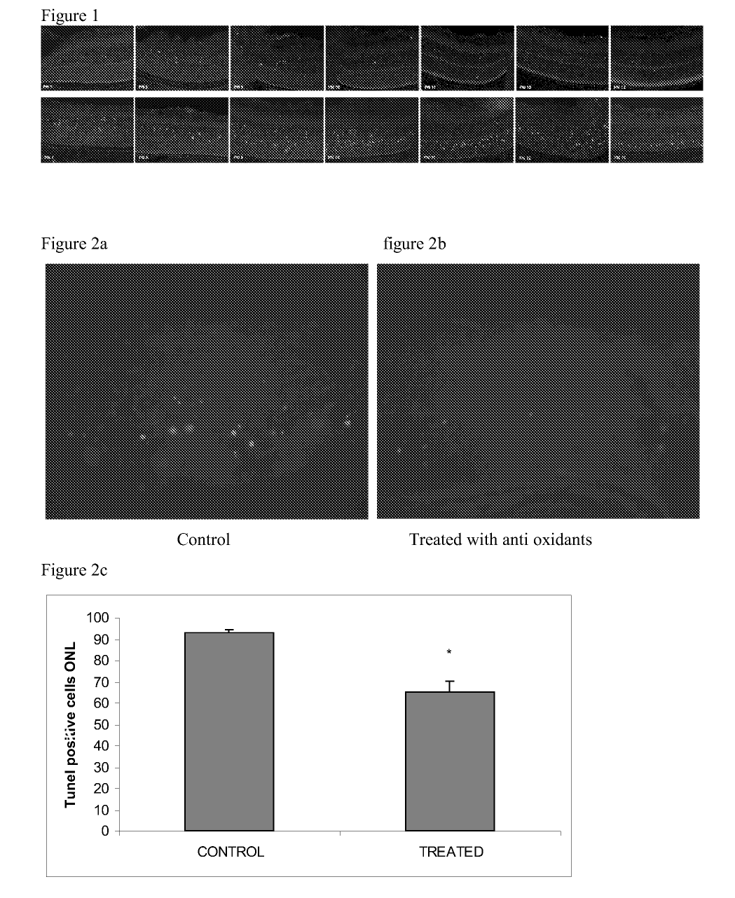

FIGS. 2a, 2b and 2c show an untreated control retina of an rd1 mouse (2a), a retina of an rd1 mouse treated according to the invention (2b), and the quantified results in a bar diagram (2c).

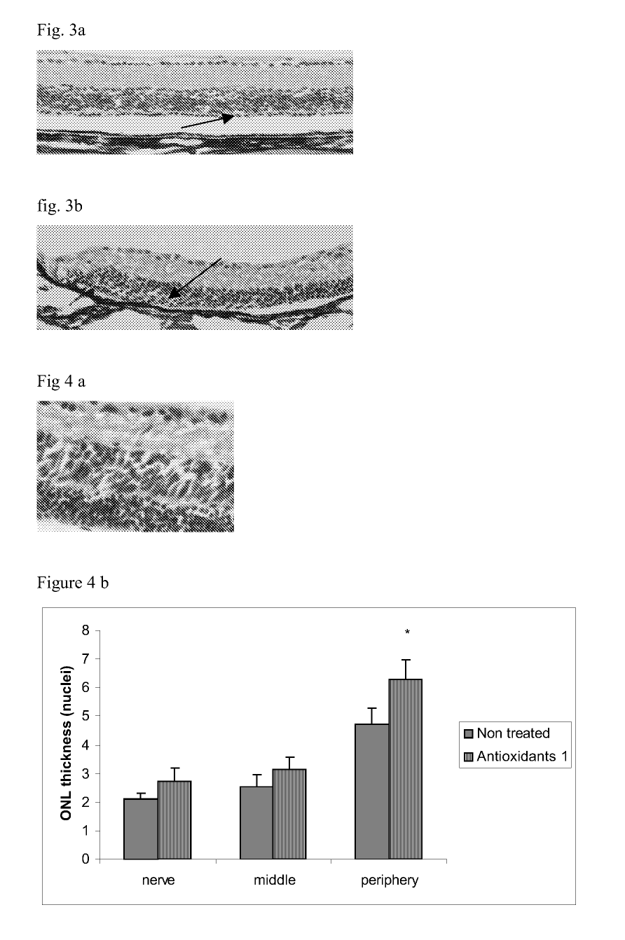

FIGS. 3a and 3b showed different regions of an untreated retina of an untreated rd1 mouse.

FIG. 4a shows a region of a treated retina, FIG. 4b shows a quantitative comparison of treated and untreated retinas.

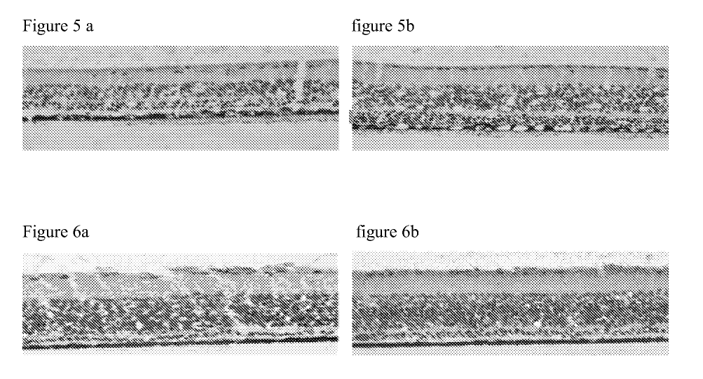

FIGS. 5a and 5b show in vitro untreated and treated retinas.

FIGS. 6a and 6b show an untreated retina and a retina treated only with glutathione.

### EXAMPLE 1

### In Vivo Experiments

The effect of the combination of lutein, zeaxanthin, glutathione and alpha lipoic acid is demonstrated below in an animal model. The animal model used here is the rd-1 mouse, which has a mutation in the gene for cGMP α-phosphodiesterase, localized in the rod photoreceptor cells in the eye. Because α-phosphodiesterase-related mutations in RP-patients play an important part in degeneration, the rd-1 mouse is a relevant and usable model. A difference is that in rd1 mice the degeneration process begins in the centre of the retina and progresses from there to the edge, while in people this takes place in opposite direction. In untreated rd-1 mice the mutation brings about apoptosis of rod receptor cells which begins on about postnatal day 10 (PN 10), and eventually leads to night blindness on about PN18. At PN18 only cone photoreceptor cells remain, which however also die off eventually. Used by way of comparison are normal wild-type mice which are genetically the same as the rd-1 mouse, but in which the mutation is absent and the degeneration of the eyesight does not occur.

### Stock Solutions

Stock solutions of zeaxanthin (Exrasynthese, France) and lutein (Exrasynthese, France) were made in the same way. 0.1-1 mg zeaxanthin or lutein was dissolved in 20 μl chloroform, supplemented with 100 μl dimethyl sulfoxide (DMSO) during vortexing. Stock solutions of L-glutathione (GSH, Sigma USA) were made by dissolving 1 or 10 mg in 2 ml water. A dosage of 2 μl/g bodyweight of this stock solution corresponds to a dosage of respectively 1 or 10 mg GSH/kg bodyweight.

1 or 10 mg α-lipoic acid (ALA, Fluka Germany) was dissolved in 100 μl DMSO.

1 to 10 mg Lucium Barbarum Lynn (LBL, Wolfberry) extract (90% polysaccharide according to UV, JF Natural, Tianjin, China) was dissolved in 2 ml water. A dosage of 2 μl/g bodyweight of this stock solution corresponds to a dosage of respectively 1 or 10 mg extract/kg bodyweight.

The stock solutions were stored at 4° C. until use. For administering purposes measured quantities of the stock solutions were dissolved homogeneously in olive oil with the desired quantity of zeaxanthin, lutein, α-lipoic acid. Glutathione, and optionally Lucium Barbarum Lynn extract, were administered as aqueous solution.

### Test Animals

The test animals were treated in accordance with the European Community guideline (86/609/EEC). Mice of the CH3 line and homozygous retinal degeneration 1 (rd1/rd1) were used. The date of birth was designated post-natal day 0 (PN0). Animals of age PN7 were sacrificed by means of decapitation, while older mice were sacrificed by suffocation with dry ice.

### Experiments

From the third day following birth (PN3) the mice were treated with an oral infusion of lutein, zeaxanthin, α-lipoic acid, glutathione and/or Lucium Barbarum Lynn-extract (LBL-extract) administered until day 11 or 16 after birth, this under different regimes as shown in table 1. Each group comprised 9 animals (n=9). The control group (1) had no lutein, zeaxanthin, α-lipoic acid, glutathione or LBL-extract administered, but only a corresponding quantity of carrier (olive oil, water). Experiments 2-6 are experiments in which treatment took place with only one of the components lutein, zeaxanthin, α-lipoic acid, glutathione or LBL-extract. Each experiment was performed in each case with both a low dosage (2a-6a) and a high dosage (2b-6b). Experiments 7a and 7b are experiments in which a combination of lutein, zeaxanthin, α-lipoic acid, glutathione and LBL-extract was administered in low (7a) and high dosage (7b).

TABLE 1
in vivo experiments (quantities in mg/kg bodyweight)

| | | | α-lipoic | | |
|---|---|---|---|---|---|
| Experiment | lutein | zeaxanthin | acid | glutathione | LBL extract |
| 1 | — | — | — | — | — |
| 2a | 0.1 | — | — | — | — |
| 2b | 1 | | — | — | — |
| 3a | — | 0.1 | | — | — |
| 3b | — | 1 | — | — | — |
| 4a | — | — | 1 | — | — |
| 4b | — | — | 10 | — | — |
| 5a | — | — | — | 1 | — |
| 5b | — | — | — | 10 | — |
| 6a | — | — | — | — | 1 |
| 6b | — | — | — | — | 10 |
| 7a | 0.1 | 0.1 | 1 | 1 | 1 |
| 7b | 1 | 1 | 10 | 10 | 15 |

Control animals (group 1) were compared to treated animals from the same nest in order to determine the effect of the treatment. Animals were killed one day after completion of the feeding period, whereafter the eyes were removed and fixed in ice-cold 4% paraformaldehyde for 2 hours, and then cut into slices of 8 μm and stained with eosin-hematoxylin. The slices were then analysed for the number of photoreceptors in 6 sections on each side of the optic nerve using the so-called ‘Terminal dUTP nick-end labelling-assay (TUNEL)’, wherein retinas of treated rd1 mice were compared to untreated rd1 mice at PN11. A commercially available cell death detection kit was herein used with the TMR-red dye (Roche Diagnostics, Mannheim, Germany). Controls consisted of omitting deoxynucleotidyl transferase from the label solution (negative control) and pretreating sections of the slices for 30 minutes with DNAse I (Roche, 3 U/ml) in 50 mM Tris-HCl buffer, pH 7.5, with 1 mg/ml for the purpose of inducing DNA damage (positive control). The negative control produced no staining, while in the positive control the whole retina was stained.

The TUNEL assay produced a weak labelling of all cell nuclei, while the rd1 outer nuclear layer (ONL) of some cells was strongly labelled. A positive labelling in TUNEL is an indication of the occurrence of apoptotic cell death in the relevant cell. The total number of TUNEL-positive nuclei in the ONL were counted in the cross-section of the central retina (3 slices on each side of the optic nerve bundle) using Zeiss Axiovision 4.2 software (Zeiss, Jena, Germany). Retinas of at least 6 rd1 test animals were measured, quantified and averaged for each experiment. Differences in the TUNEL-positive tests in rd1 treated and untreated retinas were tested statistically for relevance using an unpaired Student's t-test, wherein differences were deemed significant when p<0.05.

### Results

The results are illustrated by FIGS. 1-4. In the used animal model, the rd1 mouse, the retinal photoreceptor cells display a rapid degeneration beginning at PN9. FIG. 1 shows the development of RP in TUNEL-stained retinas of CH3H wild type and rd/rd mice, wherein apoptotically dying cells are recognisable as fluorescent dot locations. The upper row of images come from wild-type C3H mouse retinas (PN7-13), wherein no degeneration occurs apart from normal apoptosis of cells which occurs during the life of the mouse with increasing age. The retinas of the rd/rd, shown in the lower row of images of PN 7-13 from left to right, show from PN9 an increasing apoptotic death of photoreceptors cells. The dying photoreceptors cells are substantially located in the outer nuclear layer (ONL).

From this age the number of TUNEL-positive cells in the ONL increases. Treatment with the combination of the antioxidants lutein, zeaxanthin, α-lipoic acid, glutathione and LBL-extract produced a clear decrease in the number of apoptotic cells compared to the animals treated with one of the individual components and the control group (FIG. 2). FIG. 2a shows a representative example of an untreated control retina of an rd1 mouse at PN11. FIG. 2b shows a representative example of a retina of an rd1 mouse at PN11 treated with a composition according to the invention as described above. The quantified results are compared in the bar diagram in FIG. 2c. It can clearly be seen that the number of apoptotically dying photoreceptors has decreased significantly in the treated cells.

FIG. 3a shows a representative central peripheral region of the retina of an untreated rd1 mouse, wherein only one row of photoreceptors remains, indicated with the arrow. FIG. 3b shows a representative peripheral region of the same retina, wherein about three rows of photoreceptors remain. FIG. 4a shows a peripheral region of the retina of an rd1 mouse treated with a composition according to the invention, wherein about 6-7 rows of photoreceptors remain. FIG. 4b shows a quantitative comparison of the average thickness of the outer nuclear layer (ONL) of treated and untreated rd1/rd1/mice at PN 11 relative to the control. The thickness is represented as the average number of remaining rows of photoreceptors in the outer nuclear layer.

Also in the longer term (animals treated up to PN16, and analysed at PN17) the inhibiting action of the treatment according to the invention on the degeneration of the rod photoreceptor cells was found to be still noticeable, this being shown in the number of rows of photoreceptors in the peripheral retina (FIG. 3). The inhibiting effect on the degeneration can also be seen in the central peripheral and central part of the retina. Since in contrast to mice RP develops from the central part in people, the inhibition of cell death in people will be mainly noticeable precisely in the central part of the retina. The above results show the effect of the combination of the antioxidants lutein, zeaxanthin, α-lipoic acid, glutathione and LBL-extract in inhibiting the death of photoreceptor cells as a consequence of RP. The difference in activity with the individual components also shows the unexpected synergistic effect these components have. In an experiment not shown here it was found that the combination of lutein, zeaxanthin, α-lipoic acid, glutathione likewise produces an effective action and a synergistic effect. Addition of LBL-extract produces a still more significant improvement in this effect.

### Example 2

### In Vitro Experiments

### Organ Culture

The in vitro-experiments were performed on an organ culture of retina explants under serum-free conditions, as described in Caffe A R, Ahuja P, Holmqvist B, Azadi S, Forsell J, Holmqvist I, Soderpalm A K, van Veen T (2001) “Mouse retina explants after long-term culture in serum-free medium.”, J Chem Neuroanat 22: 263-273. The described technique was carried out on retina explants from rd1 mice as used in the in-vivo experiments.

Retinas of rd1 mice were transferred at PN3 to an in vitro culture on flat nitrocellulose membranes and were kept in R16 medium for 8 and 21 days, corresponding with the age PN11 and PN28 of the animals. All experiments were performed paired, wherein untreated retinas were compared to retinas which were exposed to a treatment. For the treated retinas the following concentrations were used per ml culture medium: glutathione 3 μg/ml, ALA 2 μg/ml, zeaxanthin 0.3 μg/ml, lutein 0.3 μg/ml. In order to achieve these concentrations the correct measured quantities of stock solution as described for the in vivo experiments were added to the medium. The medium was renewed every second day, whereafter the retinas were fixed in ice-cold 4% paraformaldehyde for 2 hours, and then cut into slices of 8 μm and stained with eosin-hematoxylin. The rows of photoreceptors in the outer nuclear layer (ONL) were counted in 5 slices of each composition, wherein of each slice six different sections were analysed corresponding with the sections in the in vivo experiments. The differences between treated and untreated retinas were compared statistically with Student's paired t-test, or Student's unpaired t-test, wherein differences in which p<0.05 were deemed significant.

### Results

Representative results are shown in FIGS. 5 and 6.

FIG. 5a shows H-E stained sections of untreated rd1 retinal explants, held in culture for 25 days (corresponding to PN 28 in the in vivo tests). These explants have 1-2 rows of photoreceptors in the outer nuclear layer (ONL).

FIG. 5b shows a comparable rd1 mouse explant, treated with the combination of antioxidants according to the invention, in which the outer nuclear layer (ONL) has 3-4 rows of photoreceptors.

FIGS. 6a and 6b show an example of an untreated explant and an explant treated with glutathione. The number of rows of photoreceptor cells is almost the same, although there appears to be a difference in the texture.

Table 2 shows the average number of measured rows of photoreceptor cells, wherein n is the number of explants for which the average is taken and SD is the standard deviation. The Student's t-test shows that for the combination according to the invention this is a relevant difference. Although the treatment with the individual active ingredients lutein, zeaxanthin, glutathione of alpha lipoic acid also appears according to the measurements to have some positive effect, the difference from the control group is not found to be statistically significant (according to the Student's t-test with p<0.05 as limit). This shows that the combination of substances according to the invention does indeed have a synergistic effect which is surprising in view of the lack of significant effects in the case of treatment with the individual components.

TABLE 2
the average number of measured rows of photoreceptor cells

| | Number of rows of | Difference from |
|---|---|---|
| | photoreceptors | control (Student's |
| rd/rd treatment | average ± SD, (n) | t-test) |
| Control | 1.5 ± 0.3 (n-6) | |
| Lutein | 2.0 ± 0.8 (n-6) | p > 0.05 |
| zeaxanthin | 1.9 ± 0.7 (n-6) | p > 0.05 |
| glutathione | 2.0 ± 0.9 (n-6) | p > 0.05 |
| alpha lipoic acid | 1.8 ± 0.5 (n-6) | p > 0.05 |
| Combination lutein, | 3.5 ± 0.6 (n-6) | p < 0.05 |
| zeaxanthin, | | |
| glutathione and | | |
| alpha lipoic acid | | |

### Example 3

### Forms of Administration

For use as nutritional supplement or medication for people the active substances are mixed and packed in gel capsules by means of techniques known to the skilled person. The active ingredients lutein, zeaxanthin, glutathione and alpha lipoic acid and Lucium Barbarum Lynn extract were mixed under GMP conditions, whereafter capsules were filled with the homogenous mixture. The extract used from the fruit of Lucium Barbarum Lynn has a polysaccharide content of 90%, as determined with UV. Formulation 1 comprises a relatively small quantity, wherein multiple capsules have to be taken per day in order to achieve the recommended minimal daily dosage. The capsules can be taken over the course of the day, for instance some in the morning and some in the evening. Formulation 2 comprises a high dosage, wherein 1 or 2 capsules per day are sufficient. Depending on bodyweight and a possible supplementary diet, it is possible to vary the daily dosage if desired. The compositions of the capsules according to formulations 1 and 2, and the recommended dosage and variation, are shown in table 3. As alternative embodiment it is also possible to envisage providing the individual components in separate administering units such as capsules, wherein an RP-patient takes a combination of capsules. The ingredients can here be divided for instance over two or more capsules. Formulation 3 gives an example hereof, wherein lutein and zeaxanthin are incorporated in a first capsule 3a, and the remaining components in another capsule 3b.

TABLE 3
formulations and recommended doses.

| | | | | | Recommended | Variation |
|---|---|---|---|---|---|---|
| | | | Formulation | | minimum | daily |
| | Formulation | formulation | 3 (mg) | | daily dosage | dosage |
| ingredient | 1 (mg) | 2 (mg) | 3a | 3b | (mg) | (mg) |
| Lutein | 5 | 20 | 20 | — | 20 | 10-40 |
| Zeaxanthin | 5 | 20 | 20 | — | 20 | 10-40 |
| Glutathione | 50 | 30 | — | 30 | 200 | 200-800 |
| Alpha lipoic | 20 | 70 | — | 70 | 100 | 50-300 |
| acid | | | | | | |
| extract of | 150 mg | 70 | — | 70 | 600 | 200-1000 |
| Lucium | | | | | | |
| Barbarum | | | | | | |
| Lynn | | | | | | |

The capsules consist of a capsule shell as is usual for pharmaceutical applications and nutritional supplements in which a mixture of the active ingredients is received with the composition as described above. Capsules are advantageous because the amount of additives can remain limited. It is however possible to manufacture the composition in any alternative form of administration known to the skilled person, such as pills, powders and liquid forms of administration. In the pill form or powder form a pharmaceutically acceptable excipient or filler can for instance be added to the active ingredients. Surfactants and/or emulsifiers can optionally be added in order to guarantee the homogeneity of the mixture. In the capsule form the active ingredients do not come into contact with taste organs when taken. In other forms of administration it may however be desirable to add for instance flavouring for the purpose of masking the taste of the ingredients.

### Example 4

### Treatment of Persons

A number persons suffering from degenerating vision due to various degenerative vision including age-related macular degeneration, diabetic retinopathy, retinitis pigmentosa and cataracts, administered orally at least the minimum recommended daily dosage as indicated in Table 3. Treated persons reported a slow-down and in some cases a halt in the degeneration of their vision, which was confirmed by comparing measurements taken over time. In a few cases, even an improvement of vision was reported.

Some persons who took the composition as a food supplement noticed a positive effect of on the healing of wounds and inflammation. In particular a person suffering from arthritis reported that pains and complaints were diminished. This indicates that a composition comprising pharmaceutically active quantities of lutein, zeaxanthin, glutathione and alpha lipoic acid, preferably also comprising Lucium Barbarum Lynn extract, is also useful as an anti-inflammatory agent.

1. A composition, comprising: pharmaceutically active quantities of lutein, zeaxanthin, glutathione and alpha lipoic acid.

2. The composition of claim 1, wherein the composition comprises
a) at least 1 mg lutein;
b) at least 1 mg zeaxanthin;
c) at least 20 mg glutathione; and
d) at least 5 mg alpha lipoic acid.

3. The composition of claim 1, wherein the composition comprises
a) from 1 to 40 mg lutein;
b) from 1 to 40 mg zeaxanthin;
c) from 20 to 800 mg glutathione; and
d) from 5 to 300 mg alpha lipoic acid.

4. The composition of claim 3, wherein the composition comprises
a) from 10 to 40 mg lutein;
b) from 10 to 40 mg zeaxanthin;
c) from 200 to 800 mg glutathione; and
d) from 50 to 300 mg alpha lipoic acid.

5. The composition of claim 4, wherein the composition comprises
a) from 15 to 25 mg lutein;
b) from 15 to 25 mg zeaxanthin;
c) from 180 to 220 mg glutathione; and
d) from 90 to 110 mg alpha lipoic acid.

6. The composition of claim 1, wherein lutein, zeaxanthin, glutathione and alpha lipoic acid are present in the relative weight ratio from 1 to 4 units of weight lutein, from 1 to 4 units of weight zeaxanthin, from 16 to 80 units of weight glutathione, and_from 5 to 30 units of weight alpha lipoic acid.

7. The composition of claim 1, further comprising an active quantity of Lucium Barbarum Lynn extract.

8. The composition of claim 7, wherein the composition comprises at least 20 mg Lucium Barbarum Lynn extract.

9. The composition of claim 8, wherein the composition comprises from 20 to 1000 mg Lucium Barbarum Lynn extract.

10. The composition of claim 1, wherein the composition comprises lutein, zeaxanthin, glutathione, alpha lipoic acid and LBL-extract in the following relative weight ratios:
a) from 1 to 4 units of weight lutein;
b) from 1 to 4 units of weight zeaxanthin;
c) from 16 to 80 units of weight glutathione;
d) from 5 to 30 units of weight alpha lipoic acid; and
e) from 20 to 100 units of weight Lucium Barbarum Lynn extract.

11. The composition of claim 1, wherein the composition is a nutritional supplement.

12. The composition of claim 1, wherein the composition is a medication.

13. The composition of claim 1, wherein the composition is a medication for the treatment of degenerative visual disorders.

14. The composition of claim 1, wherein the composition is a medication for the treatment of retinitis pigmentosa.

15. The composition of claim 1, wherein the composition is an anti-inflammatory medication.

16. A package, comprising: a composition having pharmaceutically active quantities of lutein, zeaxanthin, glutathione and alpha lipoic acid.
